(19) United States
(12) Patent Application Publication
(10) Pub. No.: US 2011/0052738 A1
(43) Pub. Date: Mar. 3, 2011

(54) TOPICAL PAIN FORMULATION

(76) Inventor: Gary Dean Bennett, Newport Coast, CA (US)

(21) Appl. No.: 12/812,945

(22) PCT Filed: Jan. 16, 2009
(86) PCT No.: PCT/US09/31331

§ 371 Date: Jul. 14, 2010

Related U.S. Application Data
(60) Provisional application No. 61/021,825, filed on Jan. 17, 2008.

(51) Int. Cl. A61K 36/886; A61K 31/24; A61K 36/906; A61K 31/685; A61P 29/00

(52) U.S. Cl. 424/744; 514/536; 424/756; 514/78

(57) ABSTRACT

A topical pain formulation, comprising one or more analgesic compounds and a plurality of skin penetrants.

### CROSS REFERENCE TO RELATED APPLICATIONS

This application claims priority from a United States Provisional Application filed Jan. 17, 2008, and having Ser. No. 61/021,825 filed Jan. 17, 2008, which is hereby incorporated herein by reference.

### FIELD OF THE INVENTION

This invention relates to a topical pain formulation.

### BACKGROUND OF THE INVENTION

Millions of people around the world suffer from acute and chronic pain. Chronic pain is the predominant cause of disability and lost work costing the economy of the USA billions of dollars. Treatment options for chronic pain are limited. Opiate-based medications, i.e. narcotics, have been used for centuries and are associated with habituation, tolerance, addiction, and overdose. Narcotics have a host of other ill effects such as sedation and gastrointestinal complications. A variety of non-narcotic analgesics are available for pain control, but these medications often fall short of controlling pain and also are associated with secondary effects such as liver failure, kidney failure, gastroenteritis, ulcers, and bleeding. Over-the-counter analgesic medication overdose accounts for thousands of emergency room visits each year, many associated with death. Physicians have searched for treatment options that are effective and have improved therapeutic safety margins.

Topical medications such as salicylates have enjoyed a resurgence of use because of the limited systemic secondary effects. However, a major limitation to the efficacy of topically applied medications has been the inability for active medications to pass through the thin but nearly impenetrable stratum corneum layer of the epidermis. Extensive research has been conducted in an attempt to identify the most efficient skin penetrant enhancer. Other than dimethyl sulfoxide (“DMSO”), there are few universal enhancers. The action of each enhancer is dependent on the physical and chemical properties of the active medication and the physical characteristics of the skin. The use of most of the known skin penetrant enhancers is limited by toxic effects. For example, isopropyl myristate is known to result in skin irritation and dryness, while oleic acid has been shown to damage the epidermal Langerhans cells and corneocytes which lead to a depression of T-cell immunity.

### DETAILED DESCRIPTION OF THE PREFERRED EMBODIMENTS

This invention is described in preferred embodiments in the following description. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The fundamental problem with topically applied pain medications has been the inability to penetrate the stratum corneum of the epidermal barrier to reach deeper areas of the body which may be the cause of pain such as tendons, muscles, joints, and nerves. A variety of solutions have been proposed in the past such as use of carrier agents mixed with the active pain reliever ingredient such as DMSO. Doses of DMSO up to 70% have been associated with ill effects, and can carry other topical ingredients into the blood stream at doses that may result in systemic toxicity. Mixtures of emulsifying agents known as pluronic agents have been used to improve skin penetration. Such prior art formulations comprise “sticky” preparations that are not convenient to use.

In various embodiments, Applicant's topical pain formulation utilizes one or more analgesic, anesthetic, anti-inflammatory compounds selected from the group consisting of methyl salicylate, salicylic acid, menthol, benzocaine, capsaicin, γ-linoleic acid, α-Boswellic Acid, β-Boswellic Acid, 11-keto-β-Boswellic Acid, ginger extract, Aloe Vera extract, and combinations thereof, in combination with one or more skin penetrants selected from the group consisting of soya lecithin, phosphatidyl choline, propylene glycol, derivatized propylene glycol, diethylene glycol monomethylether, diethylene glycol monoethyl ether, propylene glycol monolaurate, tri-block polyethers, isopropyl myristate, stearic acid, dimethyl sulfoxide, poloxamer 407, and combinations thereof.

In certain embodiments, Applicant's topical formulation comprises Benzocaine, compound I. Benzocaine is a local anesthetic of the ester class similar to procaine, also called Novocaine, derived from para-aminobenzoic acid, or PABA.

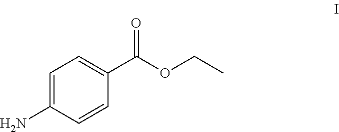

Benzocaine, like all local anesthetics, temporarily blocks the transmission of nerve impulses by interfering with the sodium channels required for normal nerve function.

Benzocaine has been safely used for many years as a topical anesthetic by dentists to numb the mouth, in throat sprays to relieve minor throat pain, and in over-the-counter preparations to block painful skin conditions such as first degree sunburns. The onset of the local anesthetic effect is very rapid, and can last for hours, providing needed pain relief. Topical benzocaine generally stays at the level of the skin. Applicant has found, however, that when combined with skin penetration enhancers, deeper penetration into underlying muscle tissue and joints is possible.

In certain embodiments, Applicant's topical formulation comprises one or more Boswellic Acids, such as α-Boswellic Acid (compound II), β-Boswellic acid (compound III), 11-keto-β-Boswellic acid (compound IV), and mixtures thereof.

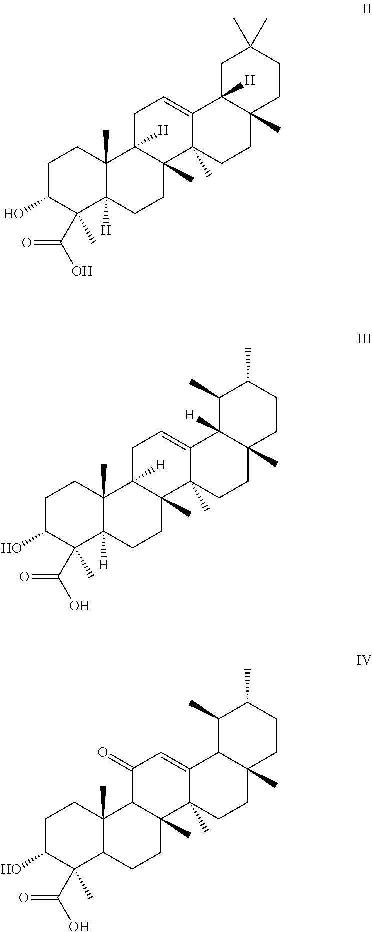

Boswellia serrata extract is derived from a tree native to India. Its gummy resin consists of essential oils, gum, and terpenoids that have been used in India for medical purposes for antiquity. The active ingredients in the terpenoid portion are boswellic acids that have been shown to have anti-inflammatory activity similar to nonsteroidal anti-inflammatory drugs and have been demonstrated to have beneficial effects in medical conditions including osteoarthritis and rheumatoid arthritis. However, unlike nonsteroidal anti-inflammatory medications which inhibit the enzyme cyclooxygenase and thereby the formation of prostaglandins, Boswellia inhibits other pro-inflammatory mediators, such as 5-lipooxygenase (5-lOX) preventing the formation of leukotrienes, which are potent inflammatory mediators. Boswellia also inhibits human leukocyte elastase (HLE). Finally, Boswellia inhibits tumor necrosis factor alpha (TNF-a), which has been identified as a powerful component in many disease states. Boswellia does not result in gastrointestinal side effects. Boswellia has been shown to prevent deterioration of cartilage and joint tissue caused by TNF-a.

In certain embodiments, Applicant's topical formulation comprises Salicylic Acid V_{A}, and/or Methyl salicylate V_{B}, and/or a salt V_{C} formed between triethanolamine and salicylic acid Methyl salicylate is a natural occurring, active component found in birch oil, wintergreen oil, gaultheria oil, and betula oil.

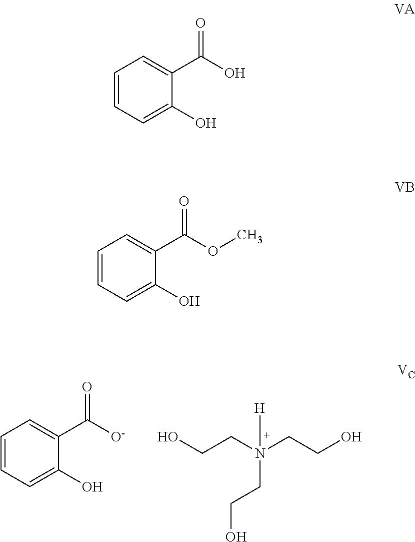

Compounds V_{B} and V_{C} belong to a larger group of very important medication called salicylates. The most widely recognized salicylate is acetylsalicylic acid, also known as aspirin.

Salicylates are powerful medications with potent anti-inflammatory and analgesic properties. The mode of action is the inhibition of the enzyme cyclooxygenase, which prevents the biosynthesis of prostaglandin, one of the most potent chemicals, which mediate pain in the body.

Salicylates are effective in relieving pain of many etiologies including headache, musculoskeletal pain, neuralgias, and arthritis. Salicylates are still recognized as the standard to which all other analgesic, anti-inflammatory medications are compared.

Applicant has found that Methyl salicylate in combination with skin penetrant enhancers passes readily through the skin and into the painful muscles and joints.

In certain embodiments, Applicant's topical formulation comprises Menthol, compound VI.

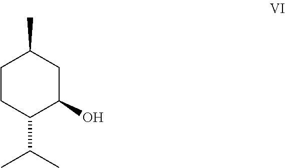

Menthol comprises anti-inflammatory properties similar to the salicylates. Menthol also suppresses pain by attaching to the peripheral nerve receptor known as transient receptor potential ion channel, known at TRPM8 causing release of substance P from the nerve endings. Substance P is the chemical which is released from pain nerve fibers during painful stimulation. Substance P has been shown to be elevated in certain medical conditions such as fibromyalgia.

Menthol is thought to act at the kappa opioid receptor to reduce pain in a similar way as narcotic medications. The release of substance P also has the effect of increasing systemic naturally occurring opioid such as enkephalins and endorphins.

In certain embodiments, Applicant's topical formulation comprises Capsaicin, compound VII, a pungent vanilloid compound, is the active ingredient in Cayenne, or Capsicum annuum, the substance contained in chili pepper, paprika, and red pepper.

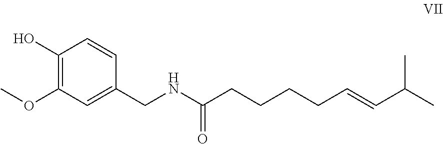

The TRPM8 receptor is different from the TRPV1 receptor, which is stimulated by capsaicin. Therefore, since two different receptors are involved, it is possible to perceive both cold and hot sensations at the same time when using menthol in combination with capsaicin. Applicant has found that menthol in combination with capsaicin act synergistically to relieve pain. Furthermore, the cooling sensation perceived with topical menthol may also ameliorate the sometimes unpleasant burning effects caused by topical capsaicin.

Capsaicin is responsible for the hot sensation in the mucous membranes when eating chili peppers. It works by attaching to the nerve receptor known as the transient receptor potential vanillod 1 (TRPV1), which activates cation channels on C nerve fibers and some A delta nerve fibers resulting in neuronal calcium influx and a sudden release of the chemical mediator in sensory nerves called substance P.

Substance P is the chemical mediator that causes the burning sensation when applied to the mucosa in chili peppers. After depletion of substance P, the sensory nerves become less sensitive to painful stimuli. When applied to the skin, the depletion of substance P results in a reduction of the severe pain which occurs in medical conditions such as diabetic neuropathy and peripheral neuropathies such as post-herpetic neuralgia or phantom limb pain. The release of substance P also has the effect of increasing systemic naturally occurring opioid such as enkephalins and endorphins. Repetitive use of capsaicin in smaller doses results in prolonged desensitization of the abnormal pain fibers. Using higher doses up to 10%, a single application for one hour may result in weeks of pain relief. Capsaicin is also effective for the treatment of painful muscle conditions, joint conditions, such as bursitis and osteoarthritis and rheumatoid arthritis.

The best results occur when capsaicin is used over an extended period, at least for several weeks, to allow time for the depletion of substance P. Applicant has found that when combined with skin penetration enhancers, capsaicin penetrates quicker and deeper into painful muscles and joints for greater effectiveness.

In certain embodiments, Applicant's topical formulation comprises antioxidant vitamins such as A, C, and E. By “Vitamin A,” Applicant means Vitamin A precursors, including one or more β-carotene, and one or more of four biologically active molecules, including, all-trans retinal (Compound VIII), 11-cis-retinal (Compound IX), retinol (Compound X), and retinoic acid, (Compound XI).

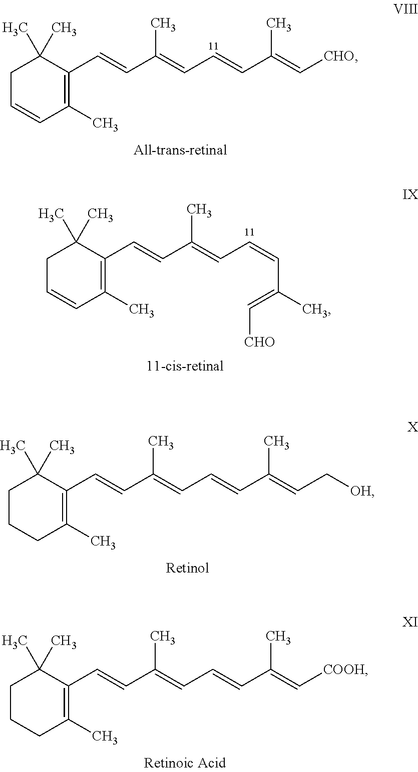

By Vitamin C, Applicant means, in certain embodiments, L-ascorbic acid, i.e. compound XII.

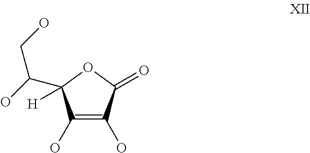

Crystalline L-ascorbic acid is reasonably stable when dry and in pure form. However, it can be readily oxidized in solution. In certain embodiments, Applicant's composition includes Vitamin C in an encapsulated form. Encapsulants include capsaicin, waxes, and combinations thereof.

In other embodiments, Applicant's composition includes derivatives of Vitamin C, such as compound XIII.

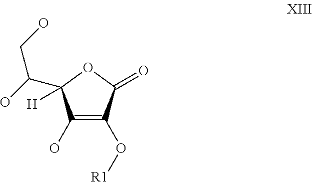

In certain embodiments, Applicant's composition includes a mono sulfate ester or a mono phosphate ester of Vitamin C, i.e. compound XIII where R1 is selected from the group consisting of a sulfate group, a phosphate group, and mixtures thereof.

In certain embodiments, Applicant's composition includes derivatizing both the 2- and 3-hydroxy groups, such as compound XIV.

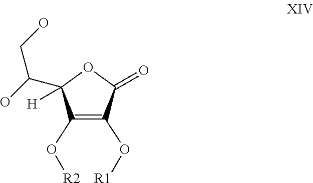

In certain embodiments, the R1 moiety of compound XIV is selected from the group consisting of hydrogen, a sulfate group, a phosphate group, and mixtures thereof. In certain embodiments, the R2 moiety of compound XIV is selected from the group consisting of hydrogen, a sulfate group, a phosphate group, and mixtures thereof. In certain embodiments, R1 and R2 are the same. In other embodiments, R1 and R2 differ.

In certain embodiments, Applicant's composition includes derivatizing the 2-, 3-, and 6-hydroxy groups, such as compound XV.

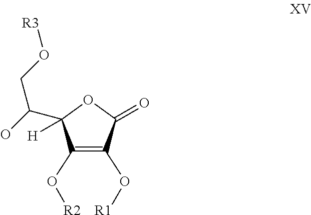

In certain embodiments, the R1 moiety of compound XV is selected from the group consisting of hydrogen, a sulfate group, a phosphate group, and mixtures thereof. In certain embodiments, the R2 moiety of compound XV is selected from the group consisting of hydrogen, a sulfate group, a phosphate group, and mixtures thereof. In certain embodiments, the R3 moiety of compound XV is selected from the group consisting of hydrogen, a sulfate group, a phosphate group, and mixtures thereof. In certain embodiments Applicant's composition includes compound XV wherein R1, R2, and R3, are the same. In other embodiments, Applicant's composition includes compound IV wherein one or more of R1, R2, and R3, differ.

In certain embodiments, Applicant's composition includes derivatizing the 2-, 3-, and 5-hydroxy groups, such as compound XVI.

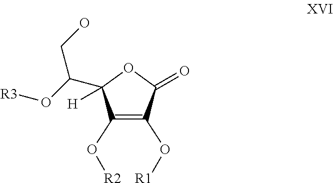

In certain embodiments, the R1 moiety of compound XVI is selected from the group consisting of hydrogen, a sulfate group, a phosphate group, and mixtures thereof. In certain embodiments, the R2 moiety of compound XVI is selected from the group consisting of hydrogen, a sulfate group, a phosphate group, and mixtures thereof. In certain embodiments, the R3 moiety of compound XVI is selected from the group consisting of hydrogen, a sulfate group, a phosphate group, and mixtures thereof. In certain embodiments Applicant's composition includes compound XVI wherein R1, R2, and R3, are the same. In other embodiments, Applicant's composition includes compound XVI wherein one or more of R1, R2, and R3, differ.

In certain embodiments, Applicant's composition includes derivatizing the 2-, 3-, 5-, and 6-hydroxy groups, such as compound XVII.

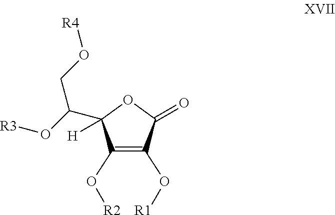

In certain embodiments, the R1 moiety of compound XVII is selected from the group consisting of hydrogen, a sulfate group, a phosphate group, and mixtures thereof. In certain embodiments, the R2 moiety of compound XVII is selected from the group consisting of hydrogen, a sulfate group, a phosphate group, and mixtures thereof. In certain embodiments, the R3 moiety of compound XVII is selected from the group consisting of hydrogen, a sulfate group, a phosphate group, and mixtures thereof. In certain embodiments, the R4 moiety of compound XVII is selected from the group consisting of hydrogen, a sulfate group, a phosphate group, and mixtures thereof. In certain embodiments Applicant's composition includes compound XVII wherein R1, R2, R3, and R4, are the same. In other embodiments, Applicant's composition includes compound XVII wherein one or more of R1, R2, R3, R4, differ.

By “Vitamin E,” Applicant means a mixture of several related compounds known as tocopherols. The α-tocopherol molecule is the most potent of the tocopherols.

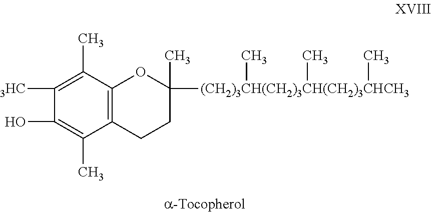

In certain embodiments, Applicant's topical formulation comprises bradykinase, an enzyme important in the breakdown of the inflammatory mediator, bradykinin. In certain embodiments, Applicant's topical formulation comprises several phytosterols such as Lupeol (compound XIX), Sisosterol (compound XX), and Campesterol (compound XXI). Applicant has found that these phytosterols also have anti-inflammatory properties and are absorbed directly through the skin.

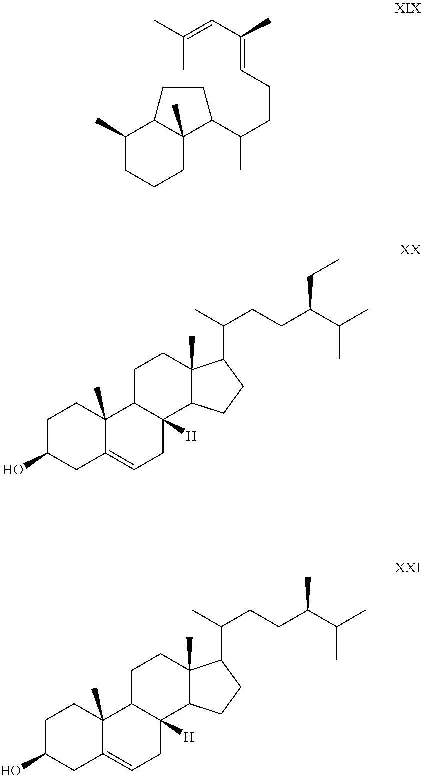

In certain embodiments, Applicant's topical pain formulation comprises Aloe Vera extracts. Aloe Vera comes from a succulent plant Aloe Vera barbadensis that has been recognized for centuries for its potent medicinal properties. Potent antiseptic and anti-inflammatory properties have been identified. Science has isolated many compounds contained in Aloe Vera with medicinal properties.

Aloe Vera comprises powerful antioxidant vitamins such as A, C, and E, minerals such as the antioxidant selenium, amino acids, vitamins, mucopolysaccarides which nourish and hydrate the skin, Salicylic acid, and the phytosterols described hereinabove.

In certain embodiments, Applicant's topical pain formulation comprises gamma-linoleic acid (“GLA”) (compound XXII).

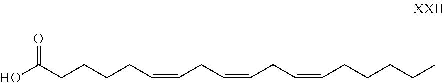

It is known in the art that in many diseases such as diabetes or inflammatory conditions, such as rheumatoid arthritis or eczema, a deficiency in essential fatty metabolism develops resulting in a deficiency of GLA, which can result in painful neuropathies. In certain embodiments, the GLA portion of Applicant's topical pain formulation comprises use of borage oil. Borage oil, also known as starflower oil and borage seed oil, is derived from the seeds of the borage plant (Barago offisinalis). The borage plant is an annual that is found in Europe and North America, North Africa, and parts of Asia.

In certain embodiments, Applicant's topical pain formulation comprises dimethyl sulfoxide (“DMSO”). DMSO has a unique ability to penetrate the cellular membrane enabling rapid absorption into the body when applied topically. Even small quantities of DMSO enhance the diffusion of other active substances through the cellular membrane to increase the cytoavailability of other therapeutic agents. In addition, DMSO possesses anti-inflammatory, analgesic, antioxidant properties, and cardioprotective effects. Recently, DMSO has been found to be effective for the relief of pain due to reflex sympathetic dystrophy or RSD, also known as complex regional pain syndrome or CRPS.

Applicant has found that certain isosorbide compounds penetrate the cellular membrane enabling rapid absorption into the body when applied topically. For example, even small quantities of isosorbide dimethyl ether, Compound XXIIIA, and/or isosorbide dinitrate, Compound XXXIIIB, enhance the diffusion of therapeutic agents through the cellular membrane to increase the cytoavailability of those therapeutic agents.

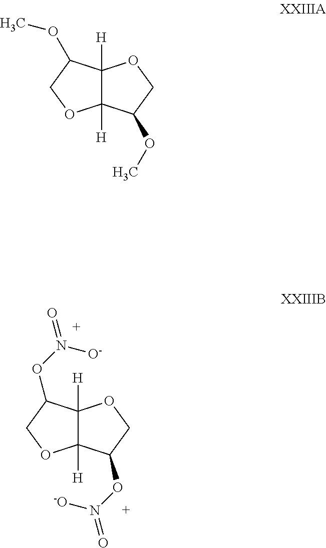

In certain embodiments, Applicant's topical pain formulation comprises ginger extracts obtained from the rhizome of Zingiber officinale. The anti-inflammatory properties of ginger extract block the production of prostaglandins which are potent pain and inflammatory mediators.

In certain embodiments, Applicant's topical pain formulation comprises phosphatidycholine, compound XXIV wherein R1 comprises an oleoyl moiety and wherein R2 comprises a palmitoyl moiety.

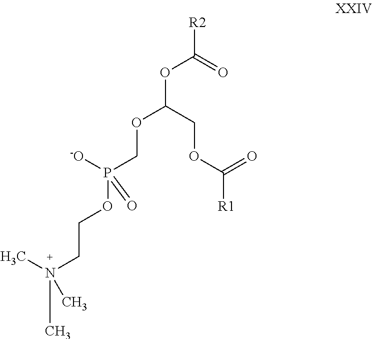

In certain embodiments, Applicant utilizes soya lecithin to his topical pain formulation to supply the phosphatidycholine.

In certain embodiments, Applicant's topical pain formulation comprises one or more di-hydroxyl compounds, i.e. diols. In certain embodiments, Applicant's topical pain formulation comprises propylene glycol XXV.

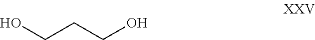

Applicant has found that propylene glycol acts to facilitate transdermal passage of the other components of his topical pain formulation. In certain embodiments, Applicant's topical pain formulation comprises propylene glycol in combination with a derivatized propylene glycol XXVI.

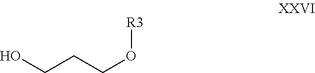

In certain embodiments, R3 is selected from the group consisting of a oleoyl moiety, a palmitoyl moiety, laurolyl moiety, and combinations thereof. In certain embodiments, Applicant's topical pain formulation comprises propylene glycol in combination with diethylene glycol monomethyl ether, i.e. methoxyethoxyethanol.

In certain embodiments, Applicant's topical pain formulation comprises a dihydroxy end-capped, block copolymer of polyethylene oxide (“EO”) end units and a polypropylene oxide (“PO”) core. In certain embodiments, the two E0 blocks comprise about 100 repeat units, and the PO block comprises about 55 repeat units. In certain embodiments, Applicant's topical pain formulation comprises a polyol sold in commerce by BASF under the trade name Lutrol F-127.

In certain embodiments, Applicant's topical pain formulation comprises Isopropyl myristate, compound XXVII.

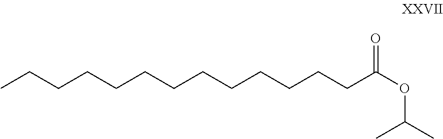

Applicant has found that isopropyl myristate facilitates transdermal passage of his topical pain formulation.

The following examples are presented to further illustrate to persons skilled in the art how to make and use the invention. These examples are not intended as a limitation, however, upon the scope of the invention.

### Example 1A

Even at low levels of 0.05 to 5%, DMSO facilitates the epidermal penetration of methyl salicylate, menthol, benzocaine, capsaicin, aloe barbadensis gel, borage oil, Boswellia serratia extract, and zingaber officinale root extract.

### Example 1B

Even at low levels of 0.05 to 5%, isosorbide nitrate facilitates the epidermal penetration of methyl salicylate, menthol, benzocaine, capsaicin, aloe barbadensis gel, borage oil, Boswellia serratia extract, and zingaber officinale root extract.

### Example 2

In this example, a mixture of triblock polyether (0.1 to 10%), soya lecithan (0.1 to 10%) and isopropyl myristate (0.1 to 10%) facilitates the epidermal penetration of methyl salicylate, menthol, benzocaine, capsaicin, aloe barbadensis gel, borage oil, Boswellia serratia extract, and zingaber officinale root extract.

### Example 3

In this example, Applicant's topical pain formulation comprises a combination of propylene glycol mono-lauryl ether, diethylene glycol monomethyl ether, propylene glycol, and stearic acid to enhance the epidermal penetration of one or more of methyl salicylate, menthol, benzocaine, capsaicin, aloe barbadensis gel, borage oil, Boswellia serratia extract, and zingaber officinale root extract.

### Example 4

In this example, Applicant's topical pain formulation comprises menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%), with and without low concentrations of DMSO (0.05-10%),

### Example 5

In this example, Applicant's topical pain formulation comprises benzocaine (2-20%), menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%), with and without low concentrations of DMSO (0.05-10%).

### Example 6

In this example, the use of capsaicin (0.01 to 5%) with benzocaine (2 to 20%) minimizes the unpleasant burning side effects.

### Example 7

In this example, Applicant's topical pain formulation comprises borage oil (0.1-10%), menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%).

### Example 8

In this example, Applicant's topical pain formulation comprises Boswellia serrata (0.1-10%) extract, menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%).

### Example 9

In this example, Applicant's topical pain formulation comprises Zingiber officinale (0.1-10%), menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%).

### Example 10

In this example, Applicant's topical pain formulation comprises capsaicin (0.01-1%), menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%), with and without low concentrations of DMSO (0.05-10%).

### Example 11

In this example, Applicant's topical pain formulation comprises Aloe Vera barbadensis (0.1-10%), menthol (1-10%), soya lecithin (0.1-10%), triblock polyether (0.1-10%), isopropyl myristate (0.1-10%), propylene glycol (0.1-10%), propylene glycol mono-lauryl ether (0.1-10%), diethylene glycol monomethyl ether (0.1-10%), and stearic acid (0.1-10%), with and without low concentrations of DMSO (0.05-10%).

### Example 12

In this example, Applicant sets forth a method to prepare one embodiment of his topical pain formulation.

### Part 1: Water Phase

| | |
|---|---|
| Distilled water | 28.13 |
| Propylene Glycol | 3.00 |
| Ginger extract | 0.40 |
| Boswellia extract | 0.10 |
| Aloe Vera powder | 0.25 |

In a first sanitized stainless steel tank, add the Part 1 ingredients. Mix for 15 minutes until uniform consistency is achieved. Titrate with triethanolamine to a pH of about 6.5-8.0. Heat to 170-175° F.

### Part 2: Oil Phase

| | |
|---|---|
| Propylene glycol monolaurate | 1.00 |
| Diethylene glycol monoethyl ether | 1.00 |
| Stearic acid | 3.20 |
| Cetyl alcohol | 2.40 |
| Methyl paraben | 0.20 |
| Propylbaraben | 0.10 |
| Lipomulase 165 | 3.60 |
| Glydant | 0.30 |
| Poloxamer 407 | 0.40 |
| DMSO-USP | 1.50 |
| Capsaicin | 0.20 |
| Borage oil | 0.10 |
| Soy lecithin | 0.50 |
| Triethanolamine | 0.90 |
| Isopropyl myristate | 0.50 |

In a second sanitized stainless steel tank, add the Part 2 ingredients. Mix for 15 minutes until uniform consistency is achieved. Titrate with triethanolamine to a pH of about 6.5-8.0. Heat to 170-175° F.

### Part 3: Microemulsification

Once both phases have reached the required temperature, add the Part 2 composition to the first tank. Hold heat at 170-175° F., and continue mixing for one hour.

### Part 4: Active Ingredients

| | |
|---|---|
| Methyl salicylate | 30.000 |
| Benzocaine | 15.000 |
| Menthol | 5.000 |

Add each active ingredient to the first tank, and mix for 15 minutes until uniform consistency is achieved. Homogenize with Siverson High-speed Rotor Stator, Quatro-71 unit, or any comparable unit, to reduce particle size to less than or equal to 1 micron. Begin cooling to 100-110° F. while gently mixing. Titrate with triethanolamine to a pH of about 6.5-8.0.

### First Clinical Study

Applicant conducted a single blinded, single cross-over, placebo-controlled comparison of Applicant's topical pain formulation with methyl salicylate, menthol, capsaicin (MMC) control.

### Patient Selection

One hundred twenty seven patients were initially selected by voluntarily agreeing to participate in the study comparing 2 different topical pain relievers with controls. All patients were referred to the pain clinic by physicians. All patients had been previously evaluated prior to participation in this study with appropriate physical examinations and appropriate laboratory testing.

Most patients had been evaluated with radiographic studies and an MRI or a CT study of the affected regions and many patients had received electrodiagnostic testing (MG/NCS). Each patient had an established diagnosis prior to participation in this study. Patients' medical conditions included degenerative disc disease of the cervical, thoracic, and lumbar spine, herniated cervical or lumbar disc, tendonopathies, bursitis, degenerative joint disease of the cervical, thoracic and lumbar spine and extremities (osteoarthritis), chronic muscular sprain/strain cervical, thoracic and lumbar spine, neuropathies (peripheral, post-herpetic neuralgia, reflex sympathetic dystrophy, and occipital neuralgia), tension headaches, and fibromyalgia.

All patients suffered with subacute and chronic pain conditions of duration ranging from 6 weeks to 6 months except for one patient with an acute herniated nucleus pulposis. No patient suffered with neoplastic disease. Patient ages ranged from 16 to 76 with the average age of 49.

All patients initially received placebo. Patients were randomly assigned to receive the control medication or the Applicant's topical pain formulation first based on the medical records number. Patients with even numbers received Applicant's topical pain formulation while patients with odd numbers received control medication. All patients then received cross-over medication after evaluation.

### Patient Evaluation

To assess the patients' subjective pain, mood, and functional levels, each patient filled out a patient questionnaire which required the patient to assess the patient's perceived level of pain, mood, and activity level using the standard numerical analogue scale (NAS) 1 to 10. The patient's functional status was determined by physically evaluating the body region involved in the patient's pain condition using tests such as range of motion. A composite functional score was given based on an average of the patient's functional score compared with accepted normal values. Patients filled out the questionnaire and were physically evaluated after application of placebo, the MMC, and the Applicant's topical pain formulation. If patients reported improvement they would be asked to determine the time of onset and duration of action. The patients were asked to note the time of onset of action and duration of action with respect to the pain relieving effects and not simply related to a notable superficial sensation caused by the medicinal agents.

### Composition of Topical Agents

The placebo was an emulsification of water, glycerin, glyceryl stearate, stearic acid, cetyl alcohol, DMDM hydantoin, methylparaben, propylparaben, and triethanolamine.

The control medication (MMC) was composed of an emulsification of 30% methyl salicylate, 5% menthol, 0.02% capsaicin, carbomer 940, glyceryl stearate, stearin acid, cetyl alcohol, polysorbate 80, DMDM hydantoin, methylparaben, propylparaben, and triethanolamine. This preparation did not contain any of the agents considered skin penetrant enhancers, benzocaine, or additional natural agents contained in the formula. This formulation was selected as a control because of its similarity to the formulations of many over-the-counter (OTC) topical pain medications available on the market at the time of this investigation. Although not clearly available on the retail market the combination of methyl salicylate and menthol with capsaicin was used as a control in an attempt to better isolate the potentially efficacious effects of the benzocaine, natural agents, and skin penetrant enhancers. Applicant's topical pain formulation (“ATPF”) was prepared according to the specification described above.

### Application of Placebo or Medications

After the initial evaluation was completed, approximately 3 grams of placebo (blinded to the patient) was applied over the painful area. The patient was reevaluated after one hour with physical evaluation and a second questionnaire. Patients were instructed to apply 3 to 5 grams (one unit dose) of either MMC control or Applicant's topical pain formulation four times daily. The follow-up appointment was scheduled between 2 days to 3 weeks. During the follow-up an additional questionnaire was filled out and the physical examination was repeated. After the follow-up, all patients then received cross-over medication. The average time of follow-up was 10 days. A second follow-up questionnaire and physical evaluation was performed between 2 days and 3 weeks (average 12 days) after the cross-over medication had been used. Average total length of follow-up: 3 weeks (range 1 to 6 weeks).

### Statistical Analysis

Statistical significance with P=0.01 was determined using the students paired t-test.

### Results

Of the 127 patients who entered the study 27 patients did not complete the study primarily because these patients were lost to follow-up or, for various reasons, could not complete the questionnaires, or failed to show to scheduled appointments in a reasonable time.

PAIN IMPROVEMENT¹

| | | | | |
|---|---|---|---|---|
| Baseline | After Placebo | After MMC | After ATPF | Formula-MMC |

PAIN LEVELS

| | | | | |
|---|---|---|---|---|
| 7.4 | 7.1 (4.0%) | 6.1 (18%)* | 3.3 (50%)* | 32%* |

ACTIVITY IMPROVEMENT

| | | | | |
|---|---|---|---|---|
| 4.4 | 4.4 (0) | 5.1 (16%)* | 6.9 (56%)* | 40%* |

MOOD IMPROVEMENT

| | | | | |
|---|---|---|---|---|
| 5.3 | 5.4 (2.0%) | 5.9 (9.2%)* | 7.5 (41%)* | 32%* |

FUNCTIONAL IMPROVEMENT

| | | | | |
|---|---|---|---|---|
| 5.3 | 5.4 (1.8%) | 5.5 (3.7%) | 7.2 (37%)* | 32%* |

| | | |
|---|---|---|
| After MMC | After ATPF | Formula-MMC |

TIME OF ONSET (HOURS)

| | | |
|---|---|---|
| 0.51 (87 pts) | 0.30 (95 pts) | 41%* |

DURATION OF ACTION (HOURS)

| | | |
|---|---|---|
| 1.8 (85 pts) | 5.1 (93 pts) | 183% or |
| | | 2.9xs* |

*P = 0.01
¹Values within ( ) reflect the relative enhancement measured using Applicant's topical pain formulation compared to placebo.

### Adverse Effects after Use of Applicant's Topical Pain Formulation

1. Burning sensation 2%;
2. Local cutaneous manifestations (pruritus and/or erythema isolated to the area of application) 1%;
3. Systemic complaints 0.

### SUMMARY OF RESULTS

1. Average subjective pain improvement with Applicant's topical pain formulation over placebo was 50%, 32% greater than with MMC.
2. Average subjective activity improvement with Applicant's topical pain formulation over placebo was 56%, 40% greater than with MMC.
3. Average subjective mood improvement with Applicant's topical pain formulation over placebo was 41%, 32% greater than with MMC.
4. Average objective functional improvement with Applicant's topical pain formulation over placebo was 37%, 32% greater than with MMC.
5. Time of onset of pain relief using Applicant's topical pain formulation 0.3 hours (18 minutes), 41% faster than the onset of MMC.
6. Duration of action of Applicant's topical pain formulation was 5.1 hours, 183% or 2.9 times longer than the duration of MMC.

### Conclusion of First Clinical Study

Compared to placebo and MMC, Applicant's topical pain formulation with multiple low concentrations of skin penetration enhancers resulted in a significantly greater improvement in pain, activity level, mood level, and objective functional achievement, with no systemic adverse reactions. Applicant's topical pain formulation with multiple low concentrations of skin penetration enhancers resulted in significantly faster onset of action and dramatically longer duration of action. No patient preferred placebo or MMC over Applicant's topical pain formulation. Every patient reported that the onset of action of Applicant's topical pain formulation was equal to or more rapid than MMC. Every patient reported that the duration of action of Applicant's topical pain formulation was equal to or greater than MMC.

### Discussion—First Clinical Study

Clinical improvement, more rapid onset of action, and greater duration of action, was achieved using benzocaine with analgesics methyl salicylate, menthol, and capsaicin, in combination with Applicant's combination of skin penetrant enhancers. In addition, the use of additional naturally occurring analgesics or anti-inflammatory agents such as ginger root extract, Boswellia, borage oil, Aloe Vera extract contributed to the improved effect of Applicant's topical pain formulation.

Significantly, the improved analgesia was noted across a wide variety of clinical disorders involving pathological conditions of muscles, nerves, joints and bones, over a wide age range of patients, and over varying regions of the body and skin types. The beneficial clinical effect of Applicant's topical pain formulation should therefore be considered non-specific to the underlying disease process.

It is noteworthy that the majority of these patients had suffered with their pain disorders for an extended period of time and most had received competent and comprehensive treatment with a wide variety of modalities prior to their referrals to Applicant's pain clinic. In most cases, these treatments included physical modalities, such as chiropractic manipulation and/or physical therapy, oral narcotic and non-narcotic analgesic medications, and other types of topically applied pain medications.

The majority of the patients were educated and most had unlimited access to medical information via the internet. It would be reasonable to conclude that the majority of these patients were sophisticated and knowledgeable about their medical conditions and the treatments available. Having failed to improve after previous treatments, including topical analgesic agents, many of these patients were skeptical regarding the potential efficacy of another topical pain reliever.

In most cases, patients clearly preferred Applicant's topical pain formulation over MMC. There were no patients who preferred MMC over Applicant's topical pain formulation. While the more rapid onset of action, extended duration of action, and greater subjective pain improvement was anticipated, both the patients and the investigators were clearly surprised regarding the magnitude of the difference between Applicant's topical pain formulation and MMC or other available OTC topical pain preparations that many of the patients had previous tried. Many patients reported that the beneficial effects were noted immediately and in some cases the patients reported complete relief of symptoms. Some patients described the effects of the formula as miraculous. The observed difference of objective functional improvement demonstrates that the plurality of skin penetration enhancing agents contained in Applicant's topical pain formulation enabled greater penetration of the active compounds into deeper tissues such as muscles, nerves, tendons, and joints which then resulted in actual functional improvement.

An evaluation of mood was initially included as a non-specific assay for the self-perceived well-being of the patient that would be easy to understand for the patient. The pronounced effect on mood by Applicant's topical pain formulation was somewhat unanticipated prior to this study. However, a majority of the patients commented on a euphoric effect after using Applicant's topical pain formulation that was not identified after using placebo or MMC control.

It appears that the patients' psychological status improved due to a release from the chronic pain and the improvement in the activity levels. In addition, the release of substance P caused by capsaicin and menthol resulted in a compensatory release of endogenous opioids such as enkephalins and endorphins.

These endogenous opioids have been linked to euphoric effects in the human population (such as the “runner's high”). The potential systemic availability of even small quantities of capsaicin or menthol after using Applicant's topical pain formulation causes a systemic release of these endogenous opioids, thereby resulting in a noticeable euphoric effect by the patient. The systemic release of such endogenous opioids likely explains the greater than anticipated analgesic effect that was observed. This euphoric effect identified after application of Applicant's topical pain formulation and not after MMC demonstrates that the skin penetration enhancing agents added to Applicant's topical pain formulation resulted in greater skin penetration of the active ingredients compared to placebo or the MMC control.

The majority of patients described pain relief within a few minutes of the application of Applicant's topical pain formulation, a few of the patients applied Applicant's topical pain formulation prior to sleep. In these cases, pain relief was described on the next day, or 8 to 12 hours later. The summation of these results skewed the final onset of action to a somewhat longer time. If these outlying times were eliminated, then the onset of action of the majority of patients would have been in the 10 to 15 minute range.

The probable deposition of active medications including benzocaine into adipose tissues and joint spaces facilitated by the synergistic effects of multiple skin penetrant enhancers, prolonged the duration of therapeutic action over MMC. Many patients reported that the effects of Applicant's topical pain formulation could be identified 24 to 48 hours later after a warm shower or with exercise involving perspiration. These patient comments suggest that the medication is deposited in the tissues such as adipose or eccrine sweat glands and later released with certain activities.

### Second Clinical Study

In a Second Clinical Study, Applicant conducted a single blinded, single cross-over, placebo-controlled comparison of Applicant's topical pain formulation with methyl salicylate, menthol, capsaicin (MMC) control. Applicant's First Clinical Study demonstrated that the use of multiple skin penetration agents resulted in an improvement of the topical pain relief product's therapeutic effectiveness when compared with the topical product containing only the medicinal portion of the product without the skin penetrant enhancers. However, that First Clinical Study did not address the whether Applicant's skin penetrant enhancers improved clinical outcome as a result of unanticipated therapeutic benefits in addition to their skin penetrant enhancing properties.

### Purpose

Applicant's Second Clinical Study was designed to determine if the ingredients which were considered to act solely as skin penetrant enhancers resulted in any therapeutic benefit when applied as a control without the medicinal agents.

### Patient Selection

Fifty (50) patients were selected from the pain service. Three patients were lost to follow up. Each patient suffered with pain over 6 months in duration. Patients were assigned randomly to one of 2 groups based on their odd or even medical records numbers. The study was performed with parallel cohort groups. Group I used a control lotion first and then the Applicant's Topical Pain formulation (“ATPF”). Group II used the ATPF first and then the control.

### Composition of Topical Agents

A control lotion (C) contained only the ingredients which have been identified herein as skin penetrant enhancers in combination with any other ingredient used for emulsification. The control lotion was not considered to have therapeutic effects. The control lotion consisting of a microemulsification of water, glyceryl stearate, PEG 100 stearate, stearic acid, propylene glycol, diethylene glycol monoethyl ether, cetyl alcohol, dimethyl sulfoxide, poloxamer 407, propylene glycol monolaurate, Isopropyl myristate, isosorbide dinitrate, Glydant, methyl paraben, propylparaben, Lipomulase 165, DMDM hydantoin, triethanolamine.

Applicant's topical pain formulation lotion contained the topical medications which are considered to have therapeutic effects and all the skin penetrant enhancing agents and emulsifying agents contained in the control lotion, referred to here as Applicant's topical pain formulation (“ATPF”). ATPF is the same therapeutic lotion tested in the First Clinical Study recited hereinabove. ATPF consisted of a microemulsification of methyl salicylate, benzocaine, menthol, capsaicin, aloe barbadensis gel, borage oil, zingibar officinale root extract, boswelia serrata extract, soya lecithin plus contents of control noted above.

### Patient Evaluation

Patients were given questionnaires describing their pain on the initial examination and at each of the follow up visits. Physicians evaluated the patients at the initial visit and follow up visits. Functional level was determined by performing range-of-motion tests of the body part(s) involved in the pain condition. Patients overall functional status was determined using a combination of the patient's report of functional changes and functional improvement determined by the physician's examination. A total functional score was obtained by averaging the patient's assessment of function and the physician's examination results. The patient's assessment of function and the physician's examination results were therefore given equal weight in the overall functional determination. Time to follow up evaluations ranged from 1 to 3 weeks. Patients were not aware of receiving either control or formula lotions.

### Medical Conditions of Each Group

Each group contained patients suffering with pain as a result of a one or a combination of a variety of medication conditions including degenerative disc disease (DDD), degenerative joint disease (DJD), reflex sympathetic dystrophy (RSD), rheumatoid arthritis (RA), fibromyalgia (Fib), neuropathies (Neurop), headache (HA), bursitis (BURS), myofascial disorders (Myofas), herniated nucleus pulposis (HNP). These medical conditions involved various anatomical locations including the upper extremities the lumbar spine (LS), the cervical spine (CS), the upper and lower extremities, chest wall, and head. There was no significant difference regarding the types of medical conditions between the two groups.

### Statistical Analysis

Statistical significance with P=0.01 was determined using the students paired t-test.

### Results

Number of females 26
Number of males 21
Average age 55 years
Age range 22-91 years

No significant differences were identified between the demographics of the two groups. In addition, no significant differences were identified between the pre or post treatment pain levels or functional levels of the two groups. Because no significant differences were identified between groups I and II, these groups were combined for the final results.

| | | | |
|---|---|---|---|
| Baseline | After Control | After ATPC | ATPC-Control |

PAIN LEVELS

| | | | |
|---|---|---|---|
| 7.9 | 7.1 (10.0%) | 4.3 (46%)* | 39%* |

FUNCTIONAL IMPROVEMENT

| | | | |
|---|---|---|---|
| 4.5 | 4.7 (4.4%) | 6.2 (38%)* | 24%* |

*value was statistically significant (P = 0.1)

Average Duration of pain relief: 3.7 hours

Range of Duration of pain relief: 1-8 hours

### Conclusion of Second Clinical Study

The beneficial therapeutic effects demonstrated in the First Clinical Study using Applicant's topical pain formulation did not result only from use of skin penetration enhancers or emulsifying agents. Rather, the therapeutic benefits of the Applicant's topical pain formulation result from a combination of a plurality of skin penetrants used in combination with a plurality of analgesics. This study, combined with previous studies which demonstrated that the use of the skin penetrants in Applicant's topical pain relief formula produced a greater benefit than use of the analgesic agents alone, confirms the efficacy of Applicant's combination of analgesic ingredients with a plurality of skin penetration enhancers.

While the preferred embodiments of the present invention have been illustrated in detail, it should be apparent that modifications and adaptations to those embodiments may occur to one skilled in the art without departing from the scope of the present invention.

I claim:

1. A topical pain formulation, comprising:
one or more analgesic compounds; and
a plurality of skin penetrants.

2. The topical pain formulation of claim 1, wherein said one or more analgesic compounds comprise benzocaine.

3. The topical pain formulation of claim 2, wherein said one or more analgesic compounds further comprise a salicylate.

4. The topical pain formulation of claim 3, wherein said one or more analgesic compounds further comprise menthol.

5. The topical pain formulation of claim 2, wherein said one or more analgesic compounds further comprise capsaicin.

6. The topical pain formulation of claim 2, wherein said one or more analgesic compounds further comprise γ-linoleic acid.

7. The topical pain formulation of claim 2, wherein said one or more analgesic compounds further comprise α-Boswellic Acid.

8. The topical pain formulation of claim 2, wherein said one or more analgesic compounds further comprise β-Boswellic Acid,

9. The topical pain formulation of claim 2, wherein said one or more analgesic compounds further comprise 11-keto-β-Boswellic Acid

10. The topical pain formulation of claim 2, wherein said one or more analgesic compounds further comprise ginger extract.

11. The topical pain formulation of claim 2, wherein said one or more analgesic compounds further comprise Aloe Vera extract.

12. The topical pain formulation of claim 2, wherein said plurality of skin penetrants further comprises phosphatidyl choline.

13. The topical pain formulation of claim 2, wherein said plurality of skin penetrants comprises isosorbide dimethyl ether.

14. The topical pain formulation of claim 2, wherein said plurality of skin penetrants comprises isosorbide dinitrate.

15. The topical pain formulation of claim 2, wherein said plurality of skin penetrants comprises DMSO.

16. The topical pain formulation of claim 15, wherein said plurality of skin penetrants further comprises soya lecithin.

17. The topical pain formulation of claim 16, wherein said plurality of skin penetrants further comprises propylene glycol.

18. The topical pain formulation of claim 17, wherein said plurality of skin penetrants further comprises diethylene glycol monomethylether.

19. The topical pain formulation of claim 18, wherein said plurality of skin penetrants further comprises isopropyl myristate.

20. The topical pain formulation of claim 19, wherein said plurality of skin penetrants further comprises stearic acid.

21. The topical pain formulation of claim 20, wherein said plurality of skin penetrants further comprises propylene glycol mono-laurate.

22. The topical pain formulation of claim 20, wherein said plurality of skin penetrants further comprises dihydroxy end-capped, block copolymer of polyethylene oxide end units and a polypropylene oxide core.
